(19) United States
(12) Patent Application Publication
(10) Pub. No.: US 2009/0087893 A1
(43) Pub. Date: Apr. 2, 2009

(54) Modified Expandase Enzyme and its use

(75) Inventors: Micheal Durairaj, Chennai (IN); Vasu Vinayagam, Chennai (IN); Vinit Tiwari, Chennai (IN); Billy Asir, Chennai (IN); Twinkle Jasmine Masilamani, Chennai (IN); Meghana Ravindranathan, Chennai (IN)

Correspondence Address: OLIFF & BERRIDGE, PLC, P.O. BOX 320850, ALEXANDRIA, VA, 22320-4850, US

(73) Assignee: Orchid Chemicals & Pharmaceutical Ltd., Chennai (IN)

(21) Appl. No.: 11/587,260

(22) PCT Filed: Apr. 20, 2005
(86) PCT No.: PCT/IB05/01040

§ 371 Date: Jan. 18, 2007

(30) Foreign Application Priority Data
Apr. 22, 2004 (IN) 366/CHE/2004
Aug. 23, 2004 (IN) 838/CHE/2004

(51) Int. Cl. C12N 9/02; C07H 21/04; C12N 15/63

(52) U.S. Cl. 435/189; 536/23.2; 435/320.1

(57) ABSTRACT

The present invention relates to a mutant penicillin expandase which comprises an amino acid substitution at one or more residue positions corresponding to those of a wild-type expandase selected from the group consisting of threonine at position 42, isoleucine at position 50, histidine at position 57, threonine at position 67, valine at position 133, threonine at position 143, proline at position 145, glycine at position 148, phenyl alanine at position 152, proline at position 196, alanine at position 240, cysteine at position 281, Serine at position 309, provided that the amino acid substitution at the residue position of cysteine at position 281 is not tyrosine.

### FIELD OF THE INVENTION

The present invention relates to a modified expandase enzyme and in particular penicillin N expandase having increased specificity for a substrate such as penicillin G.

### BACKGROUND OF THE INVENTION

β-lactam antibiotics occupy a major portion of the anti-infective segment due to low toxicity, high specificity and clinical efficacy against a wide variety of pathogenic organisms. Numerous organisms of both bacterial and fungal species produce classical β-lactam antibiotics such as penicillins, cephalosporins, cephamycins and non-classical antibiotics such as clavulanic acid and thienamycin. Penicillium chrysogenum and Cephalosporium acremonium, both of fungal family, produce Penicillin G and Cephalosporin C respectively. Streptomyces clavuligerus, a bacterium, produces the classical antibiotic cephamycin and the non-classical antibiotic clavulanic acid, which is widely known for its β-lactamase inhibition. Reviews such as (Jensen, S. E. & Demain, A. L. (1993) in Biochemistry and Genetics of Antibiotics/Biosynthesis, eds Vining, L. C & Stuttard, C., Butterworth—Heinemann, Boston) can be consulted for biosynthesis, genetic regulation and biochemical characterization of various enzymes involved in the synthesis of these antibiotics.

Many of the infectious organisms become resistant to naturally occurring penicillin antibiotics and the resistance mechanisms operate through degradation by β-lactamase, thereby requiring novel and more potent antibiotics. Cephalosporins have been remarkable in their effectiveness against resistant bacteria and hence, significant research is devoted to develop novel semisynthetic derivatives. Cephalosporin derivatives such as Cephalexin, cephadoxyl and Cefradine are produced by coupling 7-Amino deacetoxy cephalosporanic acid (7-ADCA) with appropriate side chains. Currently, 7-ADCA is produced from phenyl acetyl 7-ADCA, which in turn gets synthesized by expensive and polluting chemical processes from Penicillin G. Biotransformation, a process per se environmentally friendly and that could be cheaper than the chemical process is required.

Penicillin N expandase also known as desacetoxy cephalosporin C synthase (DAOCS), an enzyme found in species such as Streptomyces clavuligerus, Streptomyces lactamdurans, Xanthomonas lactamgenus, Flavobacterium sp, Streptomyces organanensis, Streptomyces lactamgens, Streptomyces fradiae, Streptomyces griseus and Streptomyces ofivaceus catalysing the conversion of the five membered thiazolidine ring nucleus of penicillin into the six membered dihydrothiazine nucleus of cephalosporins has become an obvious choice for an alternative route for the synthesis of cephalosporin derivatives.

Penicillin N expandase isolated from Streptomyces clavuligerus has been extensively characterized and described in Kovacevic S, et al., J. Bacteriol. 171(2): 754-760, 1989, Dotzlaf, J. E. et al., J. Biol. Chem. 264:10219-10226, 1989 and Valegard, K. et al., Nature. 394: 805-809, 1998. Penicillin N, a natural substrate of expandase, is not readily available and cleaving the adipoyl side chain is inefficient. On the other hand, Penicillin G is readily available and the phenyl acetyl side chain group can be cleaved with penicillin G amidase at high efficiency. However, Penicillin G is a poor substrate for Penicillin N expandase. Hence, commercial capitalization requires engineering the expandase and such modified expandases and their uses are described in WO01/85951, U.S. Pat. No. 6,699,699B2 and US 20030186354.

US publication No. 20030186354 discloses a mutated penicillin expandase comprising an amino acid substitution at one or more residue positions corresponding to those in a wild-type expandase selected from the group consisting of methionine 73, glycine 79, valine 275, leucine 277, cysteine 281, glycine 300, asparagine 304, isoleucine 305, threonine 91, alanine 106, cysteine 155, tyrosine 184, methionine 188 and histidine 244, provided that the amino acid substitution at the residue position of asparagine 304 is not N304L and the amino acid substitution at the residue position of cysteine 155 is C155Y.

The main objective of the present invention is to provide mutated expandase having expansion activities multiple folds higher on substrates such as Penicillin G or Penicillin V than wild-type expandase.

### DESCRIPTION OF THE ACCOMPANYING FIGURE

FIG. 1: SEQ ID NO: 1 describes the nucleotide and amino acid sequence for penicillin N expandase of Streptomyces clavuligerus.

### SUMMARY OF THE INVENTION

Accordingly, the present invention provides a mutant penicillin expandase having modified or improved ring-expanding activity. Preferably, the expandase is a Penicillin N expandase, which is modified to increase the activity on Penicillin G or Penicillin V as a substrate than wild-type expandase.

In another embodiment of the present invention, there is provided a modified expandase gene encoding the expandase mutation.

In another embodiment of the present invention, there is provided a protein having modified expandase activity.

In another embodiment of the present invention, there is provided an expression vector, which comprises a modified expandase gene.

In another embodiment of the present invention, there is provided a host strain transformed with an expression vector.

In another aspect, the invention provides a mutant penicillin expandase which comprises an amino acid substitution at one or more residue positions corresponding to those of a wild-type expandase selected from the group consisting of threonine at position 42, isoleucine at position 50, histidine at position 57, threonine at position 67, valine at position 133, threonine at position 143, proline at position 145, glycine at position 148, phenyl alanine at position 152, proline at position 196, alanine at position 240, cysteine at position 281, serine at position 309, provided that the amino acid substitution at the residue position of cysteine at position 281 is not tyrosine. In particular, the invention provides a mutated penicillin expandase which comprises one or more specific amino acid substitutions selected from the group consisting of T42A, 150V, H57R, T67A, V1331, T143S, P145L, G148E, F152L, P196S, A240T, C281R, S309P, V1331 and P196S; F152L and C281R; T42A, F152L and S309P; V133I, T143S, P196S and S309P wherein the residue positions of the amino acid substitution correspond to those of a wild-type expandase.

In another aspect, the invention provides a naturally or non-naturally occurring variant of expandase seen in organisms such as Streptomyces lactamdurans, Xanthomonas lactamgenus, Flavobacterium sp., Flavobacterium chitinovoruna, Streptomyces organanensis, Nocardia lactamdurans, Streptomyces lipmanii, Streptomyces jumonjinensis, Streptomyces wadayamensi, Streptomyces cattleya, Streptomyces laciamgens, Streptomyces fradiae, Streptomyces griseus, Streptomyces olivaceus, Strepiomyces sp. and Acremonium chrysogenum with substitutions at analogous positions disclosed in the current invention.

In another aspect, the current invention provides a variant of expandase such as expandase/hydroxylase also known as Deacetoxy/deacetylcephalosporin C synthase with significant expansion activity from organisms such as Acremonium chrysogenum. The variant can be identified by a person skilled in the art by aligning a variant with the sequence of SEQ ID No: 1. For example the equivalent amino acid to asparagine at position 304 of SEQ ID No: 1 can be identified by a person skilled in the art by aligning a variant with the sequence of SEQ ID No:1 and thus identify the equivalent residue for position 304 of SEQ ID No:1.

The modified strains of Escherichia coli DH5α containing the modified expandase genes deposited in Microbial Type Culture Collection center Chandigarh, India under Budapest treaty and were designated with the following accession numbers. MTCC 5133, MTCC 5134, MTCC 5135, MTCC 5136, MTCC 5137, MTCC 5138, MTCC 5139, MTCC 5140, MTCC 5141, MTCC 5142, MTCC 5143 deposited on Mar. 23, 2004 and MTCC 5160, MTCC 5161, MTCC 5162, MTCC 5163, MTCC 5164, MTCC 5165 & MTCC 5166 deposited on Jul. 20, 2004.

### DETAILED DESCRIPTION OF THE INVENTION

The present invention provides a mutant penicillin expandase, which shows, increased or modified ring-expanding activity preferably on Penicillin G or Penicillin V as a substrate than wild-type expandase.

A mutant penicillin expandase according to the invention comprises an expandase derived from Streptomyces clavuligerus or an expandase derived from other organisms.

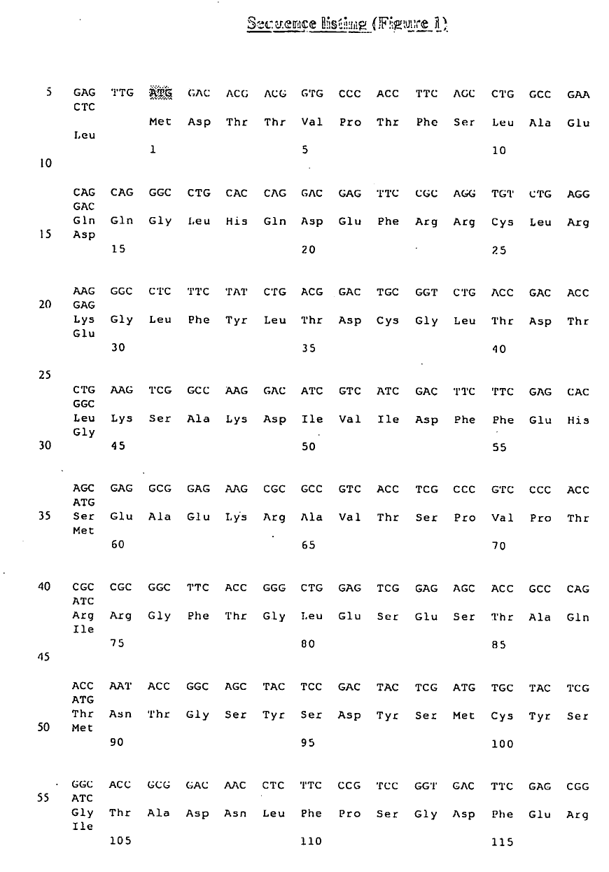

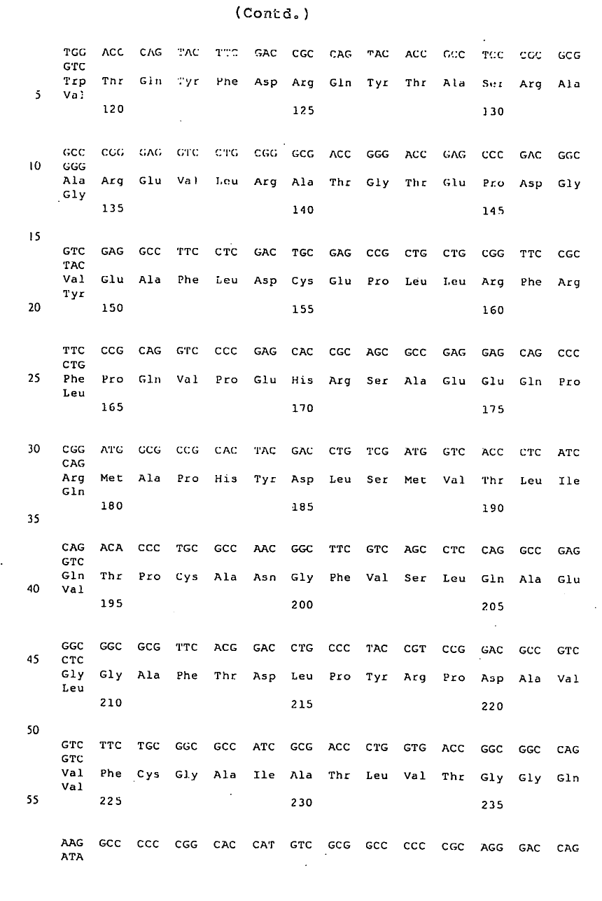

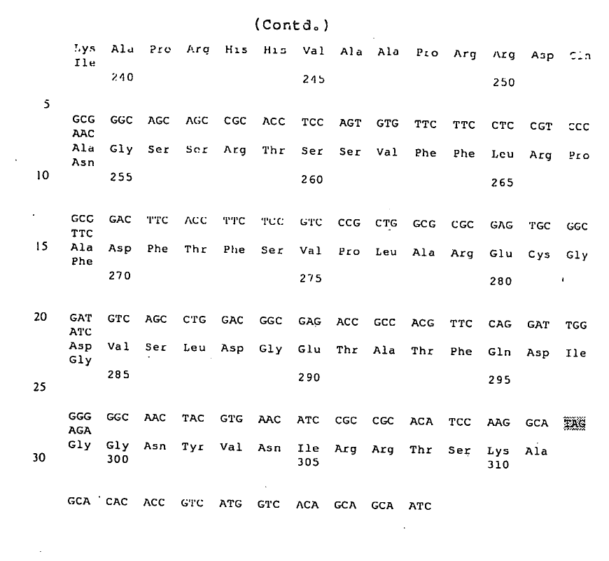

The nucleotide and amino acid sequence of penicillin N expandase from Streptomyces clavuligerus is set out in SEQ ID NO: 1 (FIG. 1)

The primary aspect of the present invention is to provide a mutated penicillin expandase having a better substrate specificity to Penicillin G or Penicillin V, wherein the mutated penicillin expandase comprises an amino acid substitution at one or more residual positions corresponding to those in a wild-type expandase selected from the group consisting of threonine at position 42, isoleucine at position 50, histidine at position 57, threonine at position 67, valine at position 133, threonine at position 143, proline at position 145, glycine at position 148, phenyl alanine at position 152, proline at position 196, alanine at position 240, cysteine at position 281, serine at position 309, provided that the amino acid substitution at the residue position of cysteine at position 281 is not tyrosine.

The variations in the sequence of SEQ ID NO. 1 are as given here:

isoleucine at position 50 is substituted by valine.

histidine at position 57 is substituted by arginine;

threonine at position 67 is substituted by alanine

proline at position 145 is substituted by leucine;

glycine at position 148 is substituted by glutamic acid;

phenyl alanine at position 152 is substituted by leucine;

alanine at position 240 is substituted by threonine

valine at position 133 is substituted by isoleucine; and proline at position 196 is substituted by serine;

phenyl alanine at position 152 is substituted by leucine and cysteine at position 281 is substituted by arginine not tyrosine;

threonine at position 42 is substituted by alanine; phenyl alanine at position 152 is substituted by leucine, and serine at position 309 is substituted by proline;

valine at position 133 is substituted by isoleucine; threonine at position 143 is substituted by serine; proline at position 196 is substituted by serine, and

serine at position 309 is substituted by proline;

histidine at position 57 is substituted by arginine, alanine at position 240 substituted by threonine;

histidine at position 57 is substituted by arginine, alanine at position 240 substituted by threonine and cysteine at position 281 substituted by arginine;

threonine at position 67 is substituted by alanine, alanine at position 240 substituted by threonine and cysteine at position 281 substituted by arginine;

isoleucine at position 50 is substituted by valine, phenylalanine at position 152 is substituted by leucine, alanine at position 240 substituted by threonine and cysteine at position 281 substituted by arginine,

histidine at position 57 is substituted by arginine, alanine at position 240 substituted by threonine and isoleucine at position 305 is substituted by methionine;

histidine at position 57 is substituted by arginine and isoleucine at position 305 is substituted by methionine,

histidine at position 57 is substituted by arginine, alanine at position 240 is substituted by threonine and cysteine at position 281 is substituted by arginine and isoleucine at position 305 is substituted by methionine;

threonine at position 67 is substituted by alanine, alanine at position 240 is substituted by threonine and cysteine at position 281 is substituted by arginine and isoleucine at position 305 is substituted by methionine.

A modified peptide in accordance with the present invention may incorporate the modifications described, for example, modification of isoleucine at position 50.

As described above, a variant polypeptide having an amino acid sequence which varies from that of SEQ ID NO: 1 may be modified in accordance with the present invention. A variant for use in accordance with the invention is one having expandase activity. A modified variant in accordance with the invention is one which demonstrates an improved ability to expand a ring substrate such as Penicillin G or Penicillin V when compared to a variant sequence not so modified.

Amino acid substitutions may be made to the amino acid sequence. One or more amino acid residues of the amino acid sequence of SEQ ID NO: 1 may alternatively or additionally be deleted. Polypeptides of the invention also include fragments of the above-mentioned sequences. Such fragments retain expandase activity.

Such fragments may be used to produce chimeric enzymes using portions of enzyme derived from other expandase polypeptides.

Polypeptides of the invention may be in a substantially isolated form. It will be understood that the polypeptide may be mixed with carriers or diluents, which will not interfere with the intended purpose of the polypeptide and still be regarded as substantially isolated. A polypeptide of the invention may also be in a substantially purified form, in which case it will generally comprise the polypeptide in a preparation in which more than 90%, e.g. 95%, 98% or 99%, by weight of the polypeptide in the preparation is a polypeptide of the invention.

The polypeptides of the invention may be introduced into a cell by in situ expression of the polypeptide from a recombinant expression vector. The expression vector optionally carries an inducible promoter to control the expression of the polypeptide.

Such cell culture systems in which polypeptides of the invention are expressed may be used in production of phenylacetyl 7-ADCA.

The present invention is illustrated with the following examples, which should not be construed for limiting the scope of the invention.

All biochemicals, reagents and oligonucleotides were obtained either from Sigma-Aldrich Chemicals Pvt. Ltd or USB, USA. Restriction enzymes and strains were purchased from New England Biolabs Inc, USA. pET24a(+) vector and BugBuster reagent were purchased from Novagen, USA. Streptomyces clavuligerus and Penicillium chrysogenum strains were obtained from ATCC.

### Cloning of Expandase Gene

Streptomyces clavuligerus (ATCC 27064) culture was grown in 50 ml of YMG medium (Yeast Extract 4 g, Malt Extract 10 g, Glucose 4 g per litre (pH7.0) in a 250 ml conical flask at 26° C. in a rotary shaker at 180 rpm until the O.D at 600 nm reached 3.00. The culture was centrifuged at 13000 rpm for 10 minutes at 20° C. to harvest the cell pellet. The cell pellet was resuspended in TE buffer pH 8.0 ( 1/10^{th} of the culture volume) containing 10 mg lysozyme (200 μl of 50 mg/ml stock) and the mixture was incubated at 30° C. for 30 min followed by the addition of 1 ml of 10% SDS, 5 ml of phenol saturated with Tris-HCl (pH 8.0) and 750 μl of 5M NaCl. The tubes containing the mixture were inverted gently a few times and kept at room temperature for 20 minutes. The suspension was centrifuged at 16,000 rpm for 10 minutes at 20° C. to separate the phases. After transferring the aqueous layer to a fresh centrifuge tube, two volumes of Isopropyl alcohol were added and the tubes were inverted gently a few times and the resulting mixture was left at room temperature for 10 minutes. It was centrifuged again at 16,000 rpm for 10 minutes at 20° C. and the pellet was resuspended in TE buffer pH 8.0. After dissolving the DNA, RnaseA was added to a final concentration of 20 μg/ml and the suspension was incubated at 50° C. for 1 hr. Subsequently, Proteinase K was added to a final concentration of 200 μg/ml followed by 100 mM NaCl and 0.4% SDS and the mixture was incubated at 37° C. for 1 hr. The suspension was again extracted with equal volume of phenol, centrifuged at 16000 rpm for 10 minutes at 20° C. and the aqueous layer was transferred to a fresh tube. The mixture was extracted similarly with chloroform and the aqueous layer was treated with Isopropanol and left at −20° C. for one hour. The DNA was precipitated by centrifuging at 13,000 rpm for 10 minutes at 20° C. and the pellet was resuspended in 50 μl TE (pH 8).

Expandase gene was amplified from Streptomyces clavuligerus (ATCC 27064) genomic DNA isolated as described above using the oligonuclotides (5′ GAGCAKATGGACACGACGGTGCCC3′, 5′GATTGCTGCTGTGACCATGACGGT3′) in a reaction volume of 100 μl containing 1× Vent DNA polymerase buffer, 10% DMSO, 1 mM MgSO₄, 2.5 units of Deep Vent DNA polymerase with an oil overlay of 50 μl. The is amplification process consisted a cycle of 5 minutes incubation at 95° C., 25 cycles of 40 seconds incubation at 95° C. for denaturation, 30 seconds incubation at 60° C. for annealing and 5 minutes incubation for extension at 72° C. followed by a final cycle of extension at 72° C. for 15 minutes. The fragment resulting from amplification was purified and cloned into SmaI restricted pUC19 vector. The sequence of the gene was further verified by sequencing.

### Generation of Mutants

Expandase gene was mutagenised using error prone polymerase chain reaction by biasing the nucleotide concentration using oligonucleotides 5′ATCGGTGCGGGCCTrCTTCGCTATTT3′, 5′CTCACTCATTAGGCACCCCAGGCT3′ in a reaction volume of 100 μl containing 1×Taq DNA polymerase buffer, 110% DMSO, 10 ng of template and 3 units of Taq DNA polymerase. The amplification process was carried out as described for cloning of expandase gene. The fragment was further purified, digested with NdeI and BamHI. It was added to similarly digested pET24a at a molar ratio of 3 to 1 and the ligation was carried out for overnight incubation at 12° C. using 0.5 mM ATP, 10× T4 DNA ligase buffer and 3 units of T4 DNA ligase. Subsequently, one μL of the ligation mix was used to transform competent E. coli BL21(DE3) prepared by CaCL₂. The recombinants were selected under kanamycin. In some cases, mutant templates were used to generate additional mutations.

### Site-Directed Mutagenesis:

Oligonucleotides incorporating the mismatches to induce desired mutations were annealed either alone or in multiple combinations to single-strand templates generated from E. coli CJ236 with the help of M13KO7 helper phage. The single-strands of native or mutant expandase gene templates were isolated by standard procedure as described in “Molecular Cloning, A laboratory Manual, 2^{nd} Edition by Sambrook et al, Cold Spring Harbor Laboratory Press, 1989. Subsequently, in vitro second-strand synthesis was carried out in presence of 200 μM of each dNTPs, 0.2 mg/ml of BSA, 0.5 mM of ATP, 2 units of T4 DNA ligase, 3 units of T4 DNA polymerase and 10 mM MgCl₂ at 42° C. for 20 minutes in a reaction volume of 20 μL. After terminating the reaction with EDTA, 1 μl was used for transformation of E. coli DH5α. Mutants were confirmed with restriction analysis followed by DNA sequencing.

### Expression of Expandase Mutants

Single colonies of E. coli BL21(DE3) harbouring putative mutant constructs were inoculated in 96 well culture plates containing LB supplemented with antibiotics and grown at 37° C. and 220 rpm. When the optical density reached between 0.6-0.8, IPTG was added to induce the expression and cultured further for 3 hours at 25° C. Subsequently, the plates were centrifuged at 4000 rpm in a microplate centrifuge and the pellet was resuspended in buffer containing 50 mM Tris HCL (pH7.5), 1 mM Dithiothreitol, 0.01 mM EDTA, 10% Glycerol and 50 mM Glucose and stored at −80° C.

### Assay of Expandase Mutants

Expression isolates were thawed on ice and treated with 100 μl of BugBuster reagent at 25° C. for 10 minutes to promote lysis of the bacteria. The expandase assay was started by adding 30 μl of freshly prepared 10× mix and 30 μl of 100 mM Penicillin G substrate to the wells, mixed, covered with breathseal and incubated at 25° C. for 30 minutes in a shaker. The final concentrations of the constituents in the mix were 50 mM ammonium sulphate, 1 mM α-ketoglutaric acid, 50 μM ascorbate, 2 mM dithiothreitol, 2 mM FeSO₄ and 10 mM Penicillin G. The reaction was quenched by adding 150 μl of CH₃OH and 150 μl of H₂O.

### Primary Screening of Expandase Mutants

25 μl of the assay mix was loaded into blank paper discs, allowed to dry and placed on LB plates containing penicillinase spread with E. coli ESS (kindly provided by Professor S. E. Jensen, University of Alberta, Canada). The plates were incubated at 37° C. for overnight and the clones with larger zone of inhibition than native expandase was short-listed for further quantification and confirmation by sequencing.

### Quantification of Expandase Activity Using HPLC

Assay samples were centrifuged for 30 minutes at 4° C. and 20 μl of it was injected and the elution profile was monitored in a C18 column using a mixture of methanol and phosphate buffer by HPLC. The conversion of Penicillin G into Cephalosporin G was quantified using Cephalosporin G as a standard and the relative activity levels for few of the expandase mutants are indicated in

Table 1.

TABLE 1
Relative Specific Activity of Expandase mutants

| | | Relative Specific |
|---|---|---|
| S. No. | Mutation | Activity (%) |
| 1 | Wild | 100 |
| 2 | A240T | 127 |
| 3 | G148E | 135 |
| 4 | T67A | 134 |
| 5 | T42A, F152L, S309P | 145 |
| 6 | HS7R | 164 |

1. A mutated penicillin expandase comprising an amino acid substitution at one or more residue positions corresponding to those in a wild-type expandase selected from the group consisting of threonine at position 42, isoleucine at position 50, histidine at position 57, threonine at position 67, valine at position 133, threonine at position 143, proline at position 145, glycine at position 148, phenyl alanine at position 152, proline at position 196, alanine at position 240, cysteine at position 281, serine at position 309, provided that the amino acid substitution at the residue position of cysteine at position 281 is not tyrosine.

2. The mutated penicillin expandase of claim 1, wherein the wild-type expandase is obtained from Streptomyces clavuligerus.

3. The mutated penicillin expandase of claim 1, comprising an amino acid substitution at one or more residue positions selected from the group consisting of T42A, 150V, H57R, T67A, V133I, T143S, P145L, G148E, F152L, P196S, A240T, C281R and S309P.

4. The mutated penicillin expandase of claim 1, comprising the amino acid substitutions of V133I/P196S; F152L/C281R; T42A/F152L/S309P; V133I/T143S/P196S/S309P, H57R/A240T, H57R/A240T/C281R, T67A/A240T/C281R, I50V/F152L/A240T/C281R, H57R/A240T/I305M, H57R/I305M, H57R/A240T/C281R/I305M and T67A/A240T/C281R/I305M.

5. A modified expandase gene encoding the expandase mutation as claimed in claim 1.

6. A modified deacetoxy/deacetylcephalosporin C synthase from Acremonium chrysogenum bearing substitutions at analogous positions described in claim 1.

7. A host strain bearing mutated penicillin expandase described in claim 1 MTCC 5133, MTCC 5134, MTCC 5135, MTCC 5136, MTCC 5137, MTCC 5138, MTCC 5139, MTCC 5140, MTCC 5141, MTCC 5142, MTCC 5143 deposited on Mar. 23, 2004 and MTCC 5160, MTCC 5161, MTCC 5162, MTCC 5163, MTCC 5164, MTCC 5165 & MTCC 5166 are deposited on Jul. 20, 2004.

8. An expression vector with mutated penicillin expandase described in claim 1.


SEQUENCE LISTING (6 sequences)

SEQ ID NO: 1 - protein, 311 aa, Streptomyces clavuligerus
  misc_feature: Amino acid sequence for penicillin N expandase of Streptomyces clavuligerus.
MDTTVPTFSLAELQQGLHQDEFRRCLRDKGLFYLTDCGLTDTELKSAKDIVIDFFEHGSEAEKRAVTSPVPTMRRGFTGLESESTAQITNTGSYSDYSMCYSMGTADNLFPSGDFERIWTQYFDRQYTASRAVAREVLRATGTEPDGGVEAFLDCEPLLRFRYFPQVPEHRSAEEQPLRMAPHYDLSMVTLIQQTPCANGFVSLQAEVGGAFTDLPYRPDAVLVFCGAIATLVTGGQVKAPRHHVAAPRRDQIAGSSRTSSVFFLRPNADFTFSVPLARECGFDVSLDGETATFQDIGGGNYVNIRRTSKA

SEQ ID NO: 2 - DNA, 975 bp, Streptomyces clavuligerus
  misc_feature: Nucleotide sequence for penicillin N expandase of Streptomyces clavuligerus.
gagttgatggacacgacggtgcccaccttcagcctggccgaactccagcagggcctgcaccaggacgagttccgcaggtgtctgagggacaagggcctcttctatctgacggactgcggtctgaccgacaccgagctgaagtcggccaaggacatcgtcatcgacttcttcgagcacggcagcgaggcggagaagcgcgccgtcacctcgcccgtccccaccatgcgccgcggcttcaccgggctggagtcggagagcaccgcccagatcaccaataccggcagctactccgactactcgatgtgctactcgatgggcaccgcggacaacctcttcccgtccggtgacttcgagcggatctggacccagtacttcgaccgccagtacaccgcctcccgcgcggtcgcccgggaggtcctgcgggcgaccgggaccgagcccgacggcggggtcgaggccttcctcgactgcgagccgctgctgcggttccgctacttcccgcaggtccccgagcaccgcagcgccgaggagcagcccctgcggatggcgccgcactacgacctgtcgatggtcaccctcatccagcagacaccctgcgccaacggcttcgtcagcctccaggccgaggtcggcggcgcgttcacggacctgccctaccgtccggacgccgtcctcgtcttctgcggcgccatcgcgaccctggtgaccggcggccaggtcaaggccccccggcaccatgtcgcggccccccgcagggaccagatagcgggcagcagccgcacctccagtgtgttcttcctccgtcccaacgcggacttcaccttctccgtcccgctggcgcgcgagtgcggcttcgatgtcagcctggacggcgagaccgccacgttccaggattggatcgggggcaactacgtgaacatccgccgcacatccaaggcatagagagcacacaccgtcatggtcacagcagcaatc

SEQ ID NO: 3 - DNA, 24 bp, Artificial Sequence
  Probe used to isolate genomic DNA from Streptomyces clavuligerus.
gagcatatggacacgacggtgccc

SEQ ID NO: 4 - DNA, 24 bp, Artificial Sequence
  Probe used to isolate genomic DNA from Streptomyces clavuligerus.
gattgctgctgtgaccatgacggt

SEQ ID NO: 5 - DNA, 24 bp, Artificial Sequence
  Oligonucleotide used to mutagenize the expandase gene by biasing the nucleotide concentration.
atcggtgcgggcctcttcgctatt

SEQ ID NO: 6 - DNA, 24 bp, Artificial Sequence
  Oligonucleotide used to mutagenize the expandase gene by biasing the nucleotide concentration.
ctcactcattaggcaccccaggct